(19) United States
(12) Patent Application Publication
(10) Pub. No.: US 2021/0128022 A1
(43) Pub. Date: May 6, 2021

(54) PHYSICAL ACTIVITY SENSOR FOR CLOTHING

(71) Applicants: Martha Lucinda Hall, Newark, DE (US); Mark Greenspan, Newark, DE (US)

(72) Inventors: Martha Lucinda Hall, Newark, DE (US); Mark Greenspan, Newark, DE (US)

(73) Assignee: University of Delaware, Newark, DE (US)

(21) Appl. No.: 16/617,356

(22) PCT Filed: Jun. 1, 2018
(86) PCT No.: PCT/US2018/035622

§ 371 Date: Nov. 26, 2019

Related U.S. Application Data
(60) Provisional application No. 62/513,459, filed on Jun. 1, 2017.

(51) Int. Cl. A61B 5/11; A61B 5/00

(52) U.S. Cl. CPC A61B 5/1118; A61B 5/6804; A61B 5/742; A61B 5/0059; A61B 5/7455; A61B 5/0022; A61B 5/7405

(57) ABSTRACT

An article of clothing that can be worn by a user to sense activity. A first conductive fabric layer is positioned in a location of the article of clothing that is deformed when worn. A second conductive fabric layer is also positioned in the location of the article of clothing. A foam layer is positioned between the first and second conductive fabric layers. At least one conductive lead is coupled to at least one of the first or second conductive fabric layers. Application of a force at the location due to activity of the user causes a change in an electrical parameter of the conductive lead.

### CROSS-REFERENCE TO RELATED APPLICATION

This application claims priority to U.S. Provisional Application No. 62/513,459, filed Jun. 1, 2017, the disclosure of which is incorporated herein by reference in its entirety for all purposes.

### FIELD OF THE INVENTION

The subject matter relates generally to a textile-based sensor integrated into an article of clothing to measure physical activity, and a method and system of using the same.

### BACKGROUND

Conventional activity monitors are usually in a bracelet or wristband form, worn around the waist, clipped onto the user, or embedded in footwear. An accelerometer type sensor is used to detect movements, such as walking or running. Typical gait patterns from the detected movements are based upon an arm swing of the user where the arm swing is analogous to, for example, a step count. Reliance on arm swing of the user can provide inaccurate results.

There exists a need to monitor activity for a user with minimum to no arm swing or an asymmetrical gait pattern.

### BRIEF DESCRIPTION OF THE DRAWINGS

The drawing figures depict one or more implementations, by way of example only, not by way of limitations. In the figures, like reference numerals refer to the same or similar elements.

FIG. 1 is a functional block diagram of an example activity monitoring system and devices, including a textile-based sensor configured to measure and provide user feedback of physical activity.

FIGS. 2A and 28 are exemplary diagrams of the textile-based sensor in a normal and deformed state in accordance with the system of FIG. 1.

FIG. 3 is a simplified block diagram of an output device such as a lighting device that operates and communicates in accordance with aspects of the invention.

FIG. 4 is a simplified diagram of a computer that may be configured as a host or server, for example, to function as the gateway or server in the system of FIG. 1.

FIGS. 5A and 58 are exemplary diagrams of the textile-based sensor integrated into an article of clothing that may be worn by a user to sense activity.

FIG. 6 is a flow chart illustrating a process of sensing physical activity using a textile-based sensor integrated into an article of clothing.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

The term “coupled” refers to any logical, optical, physical or electrical connection, link or the like by which signals produced or supplied by one system element are imparted to another coupled element. Unless described otherwise, coupled elements or devices are not necessarily directly connected to one another and intermediate components that may modify, manipulate or carry the signals may separate elements of communication media.

The term “lighting device,” as used herein, is intended to encompass essentially any type of device that processes energy to generate or supply artificial light.

The actual source of illumination light in or supplying the light for the lighting device may be any type of artificial light emitting device.

The term “artificial lighting,” as used herein, is intended to encompass essentially any type of lighting that a device produces light by processing of electrical power to generate the light. An artificial lighting device, for example, may take the form of a light fixture, lamp, or other lighting device that incorporates a light source, where the light source by itself contains no intelligence or communication capability, such as one or more LEDs or the like, or a lamp (e.g. “regular light bulbs”) of any suitable type. The illumination light output of an artificial illumination type, for example, may have an intensity and/or other characteristic(s) that satisfy an industry acceptable performance standard for a general lighting application.

The orientations of the article of clothing having an integrated textile-based sensor, associated components and/or any devices such as shown in any of the drawings, are given by way of example only, for illustration and discussion purposes.

An example article of clothing that can be worn by a user to sense activity comprises a first conductive fabric layer positioned in a location of the article of clothing that is deformed when worn, a second conductive fabric layer positioned in the location of the article of clothing, and a foam layer between the first and second conductive fabric layers. A first conductive lead is coupled to the first conductive fabric layer and a second conductive lead is coupled to the second conductive fabric layer. Application of a force at the location due to activity of the user causes a change in the resistance between the first and second leads

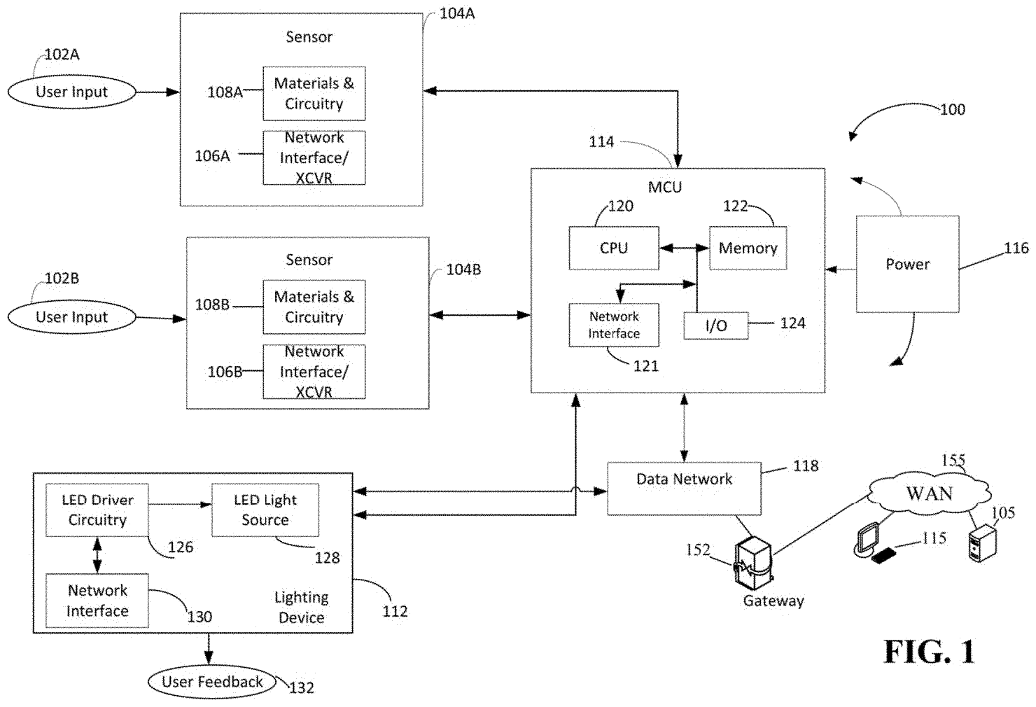

Reference now is made in detail to the examples illustrated in the accompanying drawings and discussed below. FIG. 1 is a functional block diagram of an example activity monitoring system and devices, including a textile-based sensor configured to measure and provide user feedback of physical activity.

FIG. 1 depicts an example activity monitoring system 100 using a data network 118 and devices that provide a variety of activity and user feedback capabilities, including communication of data related to the user's physical activity. Each of the devices of system 100, including a sensor 104 (e.g., a textile-based sensor), a microcontroller 114, and a lighting device 112, includes a wireless transceiver as a network interface 106/121/130 to enable data communication over the data network 118, through for example, Wi-Fi to create a local area network (LAN), Bluetooth or the like, or a wide area network (WAN) 155, via a connection to the gateway 152. Alternatively, the activity monitoring system 100 may use a wireless communication architecture in which case the data network 118 could serve as a wireless access point with each component of the system 100 being connected directly to the same network 118.

The lighting device 112 is illustrated in FIG. 1 as a single unit having a light source 128; however, a plurality of lighting devices may be provided in the activity monitoring system 100. Two sensors 104 are illustrated to receive user input 102A and 1026 from a right and left side of an article of clothing that is worn by a user to sense activity.

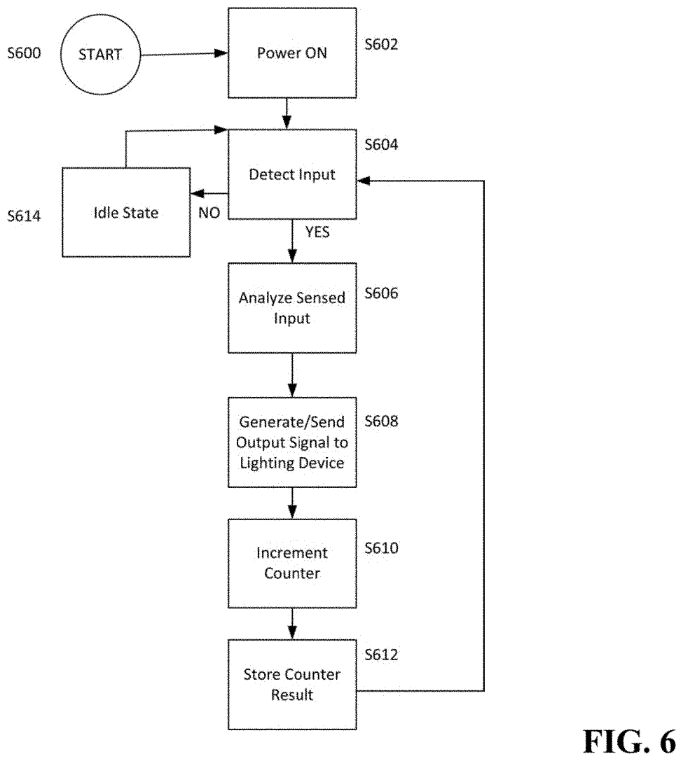

In the example of FIG. 1, the textile-based sensors 104A and 104 8 each include materials and circuity 108 configured to receive user input 102 representative of the user's physical activity. A micro-control unit (MCU) 114 is coupled to the sensors 104 and the lighting device 112. The MCU 114 is configured to communicate and process data received from each sensor 104 via the network interface/XCVR 106 as well via input/output (I/O) interface 124. MCU 114 includes a memory 122 (volatile and/or non-volatile) and a central processing unit or “CPU” 120 that are coupled to each other and the I/O interface 124 via internal data links. The CPU 120 is also configured to communicate via the network interface 121 and the network link with one or more lighting devices 112 or devices of the activity monitoring system 100, in the example, via the data network 118. MCU 114 is a programmable processor such as a central processing unit (CPU) 120 of a microcontroller or microprocessor. The memory 122 stores programming for execution by the CPU 120 of the MCU 114 and data that may be available to be processed or has been processed by the CPU 120. The memory 122, for example, may include a physical or fitness-based application (which can be firmware) for activity monitoring and data management operations. Execution of programming by the CPU 120 configures the MCU 114 to perform the functions or procedures as described below, of example, in FIG. 6. The CPU, the memory, the I/O, and the network interface of the MCU 114 typically are all included on a single chip and may be referred to as a “system on chip” or SoC.

In an implementation of the article of clothing having a textile-based sensor integrated therein, the MCU 114 may be encased in, for example, an injection molded device to allow easy removal of the MCU 114 to protect the device, for example during washing of the article of clothing.

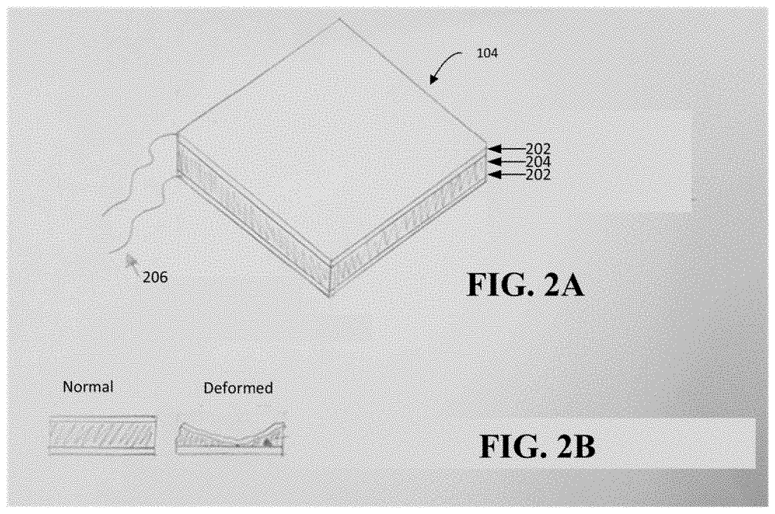

FIGS. 2A and 28 are exemplary diagrams of the textile-based sensor in a normal and deformed state in accordance with the activity monitoring system of FIG. 1. In the example illustrated in FIG. 2A, the textile-based sensor 104 includes first and second conductive fabric layers 202 and a foam layer 204 positioned between the first and second conductive layer 202. The textile-based sensor 104 is integrated into an article of clothing that can be worn by a user to sense activity. The first and second conductive fabric layers 202 are positioned in a location of the article of clothing that is deformed when worn. The first and second conductive fabric layers 202 are stretchable or non-stretchable woven fabric, non-woven fabric, or knit fabric. The fiber denier can have any value that allows the fabric to deform, for example, values between 1 and 5000. The first and second conductive fabric layers may be the same material or a different material from one another. In an example, the fabric layers 202 are knitted textile construction comprising a plurality of conductive fibers, for example, silver-plated nylon fibers. In one example, a resistance of the silver-coated fabric is less than about 1 ohm per foot in any direction. In another example, materials for the fabric layer 202 include stainless steel and graphene, where the coated fabric with any of these materials having a resistance of less than about 1 ohm per foot in any direction. Foam layer 204 is either open or closed cell construction having a thickness between 1/128″-½″. In one implementation, the foam layer 204 is open cell construction having a thickness of about ¼″, and encased in a fine tricot mesh knitted substrate.

The sensor 104 may further include at least one conductive lead 206 coupled to at least one of the conductive fabric layers 202. In the example of FIG. 2A, the textile-based sensor 104 includes first and second conductive leads 206. The conductive lead 206 may be single or multi-ply, for example, 1-4 plies of a material such as silver-coated nylon or copper wire in FIG. 2A, the first conductive lead 206 is coupled to the first conductive fabric layer 202, and the second conductive lead 206 is coupled to the second conductive fabric layer 202. When a force is applied at a surface of the first or second conductive fabric layers 202 integrated at a location of the article of clothing, a change in an electrical parameter occurs between the first and second conductive leads 206. Each of the first and second conductive leads 206 is coupled to the MCU 114 to provide analogous user input 102 received at the surface of conductive fabric layers 202. In another example, the textile-based sensor 104 may include a single conductive lead 206 coupled to at least one of the conductive fabric layers 202 to cause a change of an electrical parameter at the conductive lead 206. The electrical parameter may be a measurement of resistance between conductive leads 206 or capacitance of the single lead coupled to the either one or both conductive fabric layers 202.

In FIG. 28, the left side image illustrates the textile-based sensor 104 in a normal status, i.e., when no user input is sensed at a surface of the textile-based sensor 104. The right-side image of FIG. 2B illustrates the textile-based sensor 104 when a force is applied at the location of the article of clothing in which the textile-based sensor is integrated and which deforms the conductive fabric layers 202 and the foam layer 206.

In the example of two conductive leads coupled to the first and second conductive fiber layers illustrated in FIG. 2A, application of the force on the surface of the conductive fabric layers 202 deforms the sensor such that first and second conductive leads located along a perimeter of the conductive fabric layers 202 makes electrical contact at a point to cause a change in the resistance between the first and second leads 206. The change in resistance is proportional to the applied force at the location of the textile-based sensor 104. In an example of a single conductive lead 206 coupled to one or both of the first and second conductive fiber layers, application of a force at the location of the article of clothing due to activity of the user causes a change in conductance at the one conductive lead.

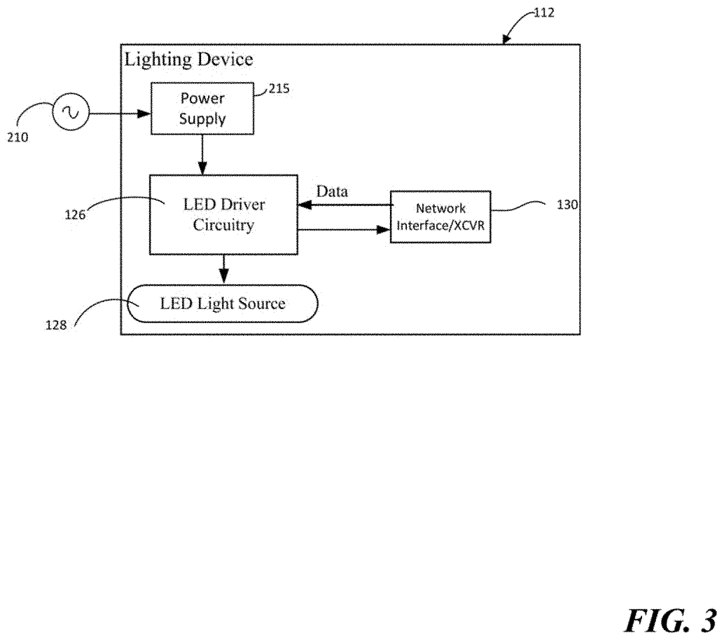

FIG. 3 is a simplified block diagram of an output device such as a lighting device that operates in and communicates in accordance with aspects of the invention. Lighting device 112 is an integrated device that generally includes a power supply 215 driven by a power source 210. Power supply 215 receives power from the power source 210 which may be powered from any AC or DC power source or MCU 114.

Lighting device 112 further includes driver circuitry, for example, LED driver circuitry 126, and a light source, which is, for example, a light emitting diode (LED) light source 128, For purposes of example, the light source 128 is illustrated and described as a LED-type light; however, the light source 128 may be virtually any type of light source suitable to providing the intended type of light output that may be electronically controlled. The LED light source 128 may include an array of LEDs, for example, RGB, arranged to provide visible feedback to the user regarding sensed physical activity. The lighting device 112 may be further configured such that the array of LEDs may also provide a haptic or audible feedback to the user regarding sensed activity. The sensed physical activity may include a stepping or running motion, or an exercise gesture, for example, deep-knee bends or arm curls. The array of LEDs may be the same color or any combination of colors.

LED driver circuitry 126 is coupled to LED light source 128 and drives the LED light source 128 by regulating the power to the LED light source 128 by providing a constant quantity or power to LED light source 128 as its electrical properties change with temperature, for example. The LED driver circuitry 126 can include an AC or DC current source or voltage source, a regulator, an amplifier (such as a linear amplifier or switching amplifier), or any other similar type of circuit or component. LED driver circuitry 126 outputs a variable voltage or current to the LED light source 128. The LED driver circuitry 126 is coupled to the LED light sources 128 and controls the LED light sources 128 based upon data or instructions received, via the network interface/XCVR 130, from the MCU 114. The LED driver circuitry may also be coupled to communicate via the network interface/XCVR 130 with one or more other lighting devices or devices of the system of FIG. 1.

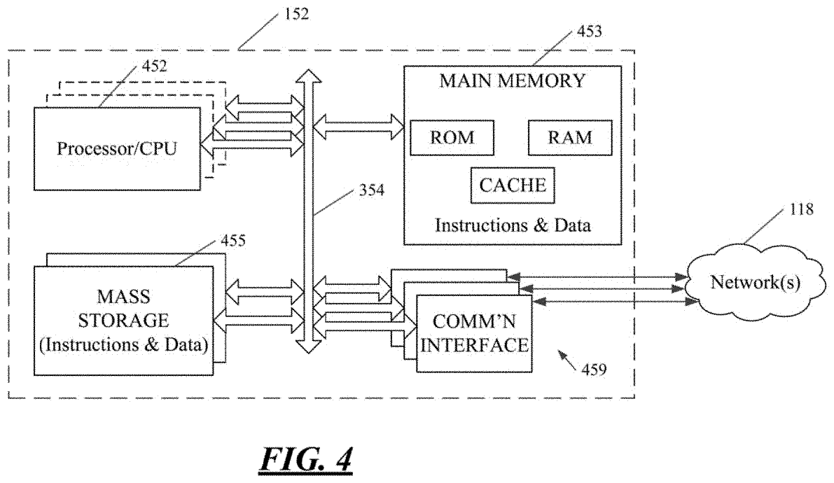

FIG. 4 is a simplified diagram of a computer that may be configured as a host or server, for example, to function as the gateway or server in the activity monitoring system of FIG. 1.

The example gateway 152 will generally be described as an implementation of a server computer. Alternatively, the computer may comprise a mainframe or other type of host computer system capable of web-based communications, media content distribution, or the like via the data network 118. The gateway 152 in the example includes a central processing unit (CPU) 452, a main memory 453, mass storage 455, and an interconnect bus 454. The circuitry forming the CPU 452 may contain a single microprocessor, or may contain a number of microprocessors for configuring the gateway 152 as a multi-processor system, or may use a higher speed processing architecture. The main memory 453, in the example, includes ROM, RAM and cache memory; although other memory devices may be added or substituted. In operation, main memory 453 is accessible to CPU 452 and stores at least portions of instructions and data for execution by the CPU 452, although instructions and data are moved between memory and storage and CPU 452 via the interconnect bus 454.

The gateway 152 also includes one or more input/output interfaces for communications, shown by way of example as interfaces 459 for data communications via the data networks 118 arid 155. Each interface 459 may be a high-speed modem, an Ethernet (optical, cable or wireless) card or any other appropriate data communications device.

The data network 118 may support data communication by equipment at the premises at a location of the system via wired, or combinations of wired and wireless technology (e.g. WiFi, Bluetooth, Zigbee, Z-wave, etc.). Such a data network 118, for example, a short range or local area network (LAN), also is configured to provide data communications for at least some of the textile-based sensors 104 and other equipment at the premises, and via a data network 155 outside the premises, shown by way of example in FIG. 1 as a wide area network (WAN), so as to allow the textile-based sensor 104 and MCU 114 at the premises to communicate with outside devices such as the server/host computer 105 and the other terminal device 115. One of ordinary skill in the art will recognize other types of suitable outside devices such as mobile devices inclusive of cellular phones, tablets, or laptop computer devices.

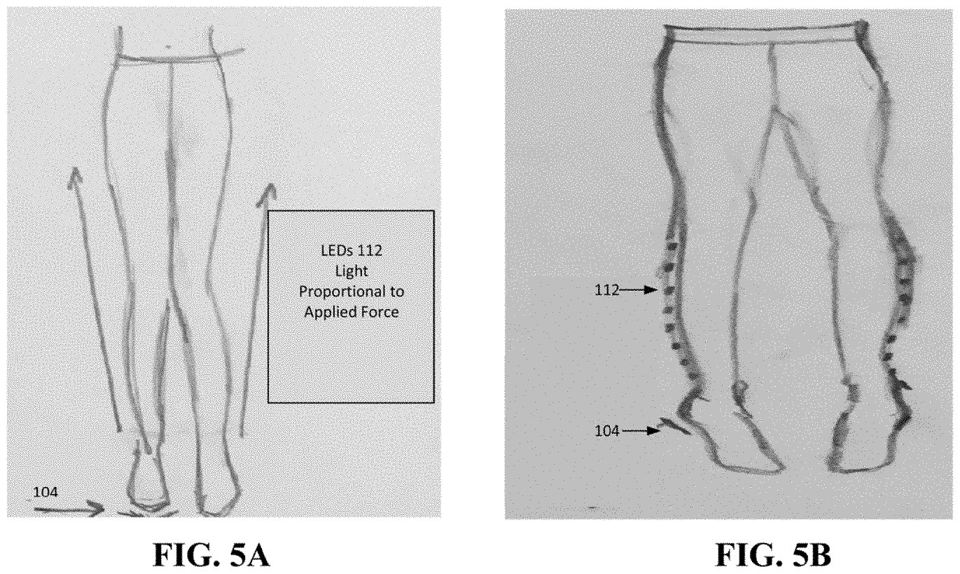

FIGS. 5A and 5B are exemplary diagrams of the textile-based sensor integrated into an article of clothing that may be worn by a user to sense activity. In FIGS. 5A and 5B, the article of clothing is, for example, a pair of pants including stirrups and the location of the textile-sensor is within the stirrup. FIG. 5A illustrates an example of the article of clothing being pants including a stirrup in which the textile-based sensor 104 is located within the stirrup and under a front part of a user's foot. Although FIG. 5A illustrates one textile-based sensor located under a right foot of the user, it is understood that each foot or side of the pants would include a textile-based sensor 104 configured to sense activity for each of the right and left sides. As illustrated in FIG. 5A, an array of LEDs 112 may illuminate in a pattern along the sides of the pants in a manner that is proportional to the applied forced sensed during physical activity of the user, e.g., proportional to the deformation of the textile-based sensor 104 when the user's foot steps down, The pattern of illumination may be defined by the user to provide notification or visual feedback of pre-set goals, for example, a step count, gait measurements, distance, etc. In addition to visual feedback, the array of LEDs 112 may be configured to provide haptic or audible feedback to the user regarding the pre-set goals.

FIG. 5B illustrates an example of the article of clothing including a stirrup in which the textile-based sensor 104 is located within the stirrup and under a heel of the user's right foot. Although not illustrated, a textile-based sensor is located within the left stirrup of the pants. Similar to the description above, an array of LEDs 112 may illuminate in a pattern along the rear or backside of the pants in a manner that is proportional to the applied force sensed during physical activity of the user. The pattern of illumination may be defined by the user to provide notification or visual feedback of pre-set goals, for example, a step count, gait measurements, distance, etc. In addition to visual feedback, the array of LEDs 112 may be configured to provide haptic or audible feedback to the user regarding the pre-set goals.

FIG. 6 is a flow chart illustrating a process of sensing physical activity using a textile-based sensor integrated into an article of clothing. The process starts at step S600. A description of the flowchart with reference to the textile-based sensor integrated into an article of clothing that can be worn by a user to sense activity follows.

At S602, the MCU 114 is powered “on.” In an example, the MCU 114 supplies power to each of the components of the activity monitoring system of FIG. 1, including the textile-based sensors 104 and the lighting device 112. Upon powering “on,” the MCU 114 may access and open an application(s) such as a fitness or exercise application from a memory 122.

At S604, MCU 114 communicates with the textile-based sensors 104 to determine whether user input 102 is detected. If user input 102 is detected or sensed at a surface of the textile-based sensor, then the process moves S606. If no user input 102 is detected, then the process moves to S614; an idle state, to await a detection of user input 102.

At S606, the sensed user input is analyzed by the MCU 114. The detected user input 102 represents a force applied at a location of the article of clothing having the textile-based sensor integrated at the location. The applied force is received at the location in the article of clothing including the textile-based sensor where the textile-based sensor includes a first conductive fabric layer and a second conductive layer separate from the first conductive layer by a foam layer. The applied force causes a change in resistance between a first lead coupled to the first conductive fabric layer, and a second lead coupled to the second conductive fabric layer. During the analysis of the received applied force, the change in resistance between the first lead and the second lead is measured. In an example, the change in resistance is analogous to a set when walking or running, or an exercise gesture, for example, with deep-knee bending or squatting, or arm curls. The measured change in resistance between the first lead and the second lead would result in this example would provide a count(s) of physical activity. In another example, the change in resistance is measured to determine, for example, a gait pattern that can be used to provide a distance measurement. A particular type of activity may be determined based upon the measured change in resistance. For example, a count of physical activity is indicative of walking, running or exercise gestures. The MCU 114 generates and transmits data to the lighting device 112 to provide instructions of display of an illumination pattern by the array of LEDs 128. The process continues to S610.

At S610, a counter is incremented for each measurement of change in resistance. The counter value and related data, for example, time/date stamp information, is stored in a memory, for example, 122, at S612. After the counter result is stored, the process returns to S604 to await detection of user input 102. When no user input is detected, the system 100 remains in an idle state at S614 until a new user input is detected or the system is powered “off.”

The term “application” or “applications” refers to program(s) that execute functions defined in the programs. Various programming languages can be employed to create one or more of the application, structured in a variety of manners, such as, object-oriented programming languages (e.g., Objective-C, Java, or C++) or procedural programming languages (e.g., C or assembly language). In one implementation, a third party application (e.g., an application developed using the ANDROID™ or IOS™ software development kit (SDR) by an entity other than the vendor of the particular platform) may be mobile software running on a mobile operating system.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus the following claims are hereby incorporated into the Detailed Description, with each claim standing on its own as a separately claimed subject matter.

While the foregoing has described what are considered to be the best mode and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An article of clothing that can be worn by a user to sense activity, the article of clothing comprising:
a first conductive fabric layer positioned in a location of the article of clothing that is deformed when worn;
a second conductive fabric layer positioned in the location of the article of clothing;
a foam layer positioned between the first and second conductive fabric layers;
at least one conductive lead coupled to at least one of the first or second conductive fabric layers,
wherein application of a force at the location due to activity of the user causes a change in an electrical parameter of the conductive lead.

2. The article of clothing of claim 1, wherein the at least one conductive lead is coupled to at least one of the first or second conductive fabric layer, and the electrical parameter is capacitance.

3. The article of clothing of claim 1, wherein a first of the at least one conductive lead is coupled to the first conductive fabric layer, a second of the at least one conductive lead is coupled to the second conductive fabric layer, and the electrical parameter is a change in resistance between the first and second conductive leads.

4. The article of clothing of claim 1, wherein each of the first and second conductive fabric layers are one of a woven fabric, a non-woven fabric, or a knit fabric.

5. The article of clothing of claim 4, wherein a fiber denier of the woven, non-woven or knit fabrics is between 1 and 5000.

6. The article of clothing of claim 1, wherein the first and second conductive fabric layers comprise one or more conductive fibers coated on a surface of the fabric layers.

7. The article of clothing of claim 6, wherein at least one of the conductive fibers is a tin, nickel, copper, silver, or steel coating or plating over a synthetic or synthetic blend fiber.

8. The article of clothing of claim 7, wherein a resistance of the conductive fabrics having the conductive fibers thereon is less than 1 ohm per foot.

9. The article of clothing of claim 4, wherein each of the first and second conductive fabric layers are a stretchable or non-stretchable fabric.

10. The article of clothing of claim 1, wherein the change in resistance is proportional to the applied force at the location,

11. The article of clothing of claim 1, wherein the article of clothing is a pair of pants including a stirrup and the location is within the stirrup.

12. The article of clothing of claim 1, wherein the foam layer is open-cell construction and has a thickness between about 1/128 inch and about ½ inch.

13. The article of clothing of claim 12, wherein the foam layer has a thickness of ¼ inch and is encased in a tricot mesh knitted substrate.

14. The article of clothing of claim 1, further comprising an output device coupled to the first and second leads, the output device positioned in another location in the article of clothing that is spaced from the location.

15. A method for sensing physical activity using a textile-based sensor integrated into an article of clothing that can be worn by a user, the method comprising:
receiving a force applied at a location in the article of clothing including a first conductive fabric layer and a second conductive fabric layer separated from the first conductive fabric layer by a foam layer, wherein the applied force causes a change in an electrical parameter at least one conductive lead coupled to at least one of the first or second conductive fabric layers;
measuring a change in the electrical parameter of the at least one conductive lead coupled to the first or second conductive fabric layers;
determining a physical activity action from the measured change in the electrical parameter; and
transmitting a signal to an output device.

16. The method of claim 15, wherein the electrical parameter is one of resistance or capacitance.

17. The method of claim 15, further comprising incrementing a counter for each physical activity action.

18. The method of claim 17, further comprising storing a result of the counter in a memory.

19. The method of claims 15, wherein the physical activity action is at least one of a stepping motion, running motion, or an exercise gesture.

20. The method of claim 15, wherein the first and second conductive fabric layers comprise one or more conductive fibers.

21. The method of claim 20, wherein at least one of the conductive fibers is silver-plated nylon or stainless steel.

22. The method of claim 15, wherein a feedback to the user is at least one of visual, haptic or audible.

23. A system for measuring physical activity, comprising:
a data network;
an article of clothing comprising:
at least two textile-based sensors, each textile-based sensor comprising:
a first conductive fabric layer positioned in a location of the article of clothing that is deformed when worn;
a second conductive fabric layer positioned in the location of the article of clothing;
a foam layer positioned between the first and second conductive fabric layers; and
at least one conductive lead coupled to at least one of the first or second conductive fabric layers, wherein application of a force at the location due to activity of a user of the article of clothing causes a change in an electrical parameter at the at least one conductive lead;
a lighting device, comprising:
a plurality of light sources;
driver circuitry coupled to the light sources to provide power to the light sources; and
a network interface to enable the lighting device to receive communication via the data network;
a processor including a network interface and coupled, via the at least one conductive lead of the textile-based sensors, to receive signals based upon user input at a surface of at least one of the textile-based sensors;
memory coupled to be accessible to the processor; and
programming in the memory, wherein the memory stores the programming for execution by the processor and data to be saved or processed by the processor during execution of the programming,
wherein execution of the programming in the memory configures the processor to:
analyze the signals based upon the user input received from the at least one textile-based sensor;
generate a user feedback signal based upon the analyzed signals; and
transmit the user feedback signal to the lighting device to control an output from the plurality of light sources.

24. The system of claim 23, wherein the execution of the programming by the processor to perform the function to analyze the signals based upon the received user input includes:
ascertaining from which of the textile-based sensors the user input is received;
measuring the change in the electrical parameter;
determining a physical activity action from the measured change in the electrical parameter; and
incrementing a counter for each physical activity action.

25. The system of claim 23, wherein a first conductive lead is coupled to the first conductive fabric layer, a second conductive lead is coupled to the second conductive fabric layer, and the electrical parameter is a change in resistance between the first and second leads.

26. The system of claim 23, wherein the at least one conductive lead is coupled to at least one of the first or second conductive layer, and the electrical parameter is capacitance.
